(19) United States
(12) Patent Application Publication
(10) Pub. No.: US 2019/0178881 A1
(43) Pub. Date: Jun. 13, 2019

(54) Virus-like Particle Bound Magnetic Particles as Reagents for Immunodiagnostics

(71) Applicant: The Government of the United States, as Represented by the Secretary of the Army, Fort Detrick, MD (US)

(72) Inventors: Keersten M. Ricks, Frederick, MD (US); Charles J. Shoemaker, Stephen's City, VA (US); Randal J. Schoepp, Frederick, MD (US); Connie S. Schmaljohn, Middletown, MD (US)

(21) Appl. No.: 16/178,635

(22) Filed: Nov. 2, 2018

Related U.S. Application Data
(60) Provisional application No. 62/581,023, filed on Nov. 2, 2017.

(51) Int. Cl. G01N 33/543; G01N 33/531; G01N 33/569; G01N 33/68

(52) U.S. Cl. CPC G01N 33/54326; G01N 33/531; G01N 2446/20; G01N 33/6854; G01N 2800/26; G01N 33/56983

(57) ABSTRACT

Described herein are virus-like particles (VLPs) that were generated to present the viral glycoprotein antigens on their surface, coupled to magnetic fluorescent microspheres to create VLP-conjugated microspheres (VCMs) and methods of making them. These VCMs were stable when lyophilized and stored at 37° C. and able to detect antibodies in non-human primate (NHP) and human clinical sera at dilutions of 1×10⁵ and 1×10⁴, respectively. Methods are also disclosed for detecting an immune response using VCMs.

### CROSS REFERENCE TO RELATED APPLICATION

The present application claims the benefit of U.S. Provisional application Se. No. 62/581023 filed Nov. 2, 2017, the contents of which are incorporated by reference herein in their entirety.

### STATEMENT AS TO RIGHTS OR INVENTIONS MADE UNDER FEDERALLY SPONSORED RESEARCH AND DEVELOPMENT

This invention was made with government support from the Medical Research Institute of Infectious Diseases, a subordinate organization of the United States Army Medical Research and Materiel Command. The United States government has certain rights in the invention.

### BACKGROUND

The spread of infectious disease continues to present a challenge for global public health initiatives. Recent evidence includes the recent Ebola outbreak in West Africa and Zika outbreak in the Americas (5, 9). Diagnostic tools for early detection and identification of viral infectious diseases must be (a) specific for the targets of interest, since early symptoms of infection can be confounding, (b) broadly applicable in order to target and identify emerging disease biomarkers, and (c) accessible-by and stable-in the diagnostic setting, in order to inform treatment and promote surveillance at the point-of-care (3). Generally, an orthogonal system that uses sensitive and specific molecular assays in combination with immunological methods (e.g. ELISA) that are less sensitive, but more broadly reactive than PCR, provides the highest confidence in a diagnostic result (1, 23-24). In this way, the diagnostic system has the greatest chance of detecting a new or re-emerging pathogen.

Enveloped viruses comprise the vast majority of pathogenic viral diseases affecting human populations. Viral glycoproteins present on the surface of these viruses represent a useful antigen target for detecting host serological responses to pathogenic viruses, as they typically elicit robust antibody responses. Anti-glycoprotein responses are usually evaluated via direct immunoassay methods, using recombinant protein, inactivated virus, or inactivated lysates from infected cells to capture any viral antibodies in a sample. However, use of whole virus or inactivated virus presents challenges for use in immunoassays. These assays must be performed in containment (when using whole virus) or with inactivated virus (which could destroy epitopes after inactivation protocols) (10). Recombinant proteins arguably are the most sustainable. They do not require BSL3/4 facilities for production, isolation, and use. However, soluble recombinant proteins come with substantial caveats, including: (i) the need for truncation to facilitate soluble release of the recombinant protein, (ii) artificial protein structure due to the lack of a membrane anchor, and/or (iii) complete resistance to recombinant expression due to heterodimeric or complex maturation behavior (13, 15-16, 26). An ideal solution involves a sustainable BSL-2 diagnostic reagent for these viral antigens that presents them to analyte material (i.e. whole blood or sera) in the context of a viral envelope. In lieu of authentic virus, two primary strategies exist for presenting glycoproteins on a safe heterologous, background particle: (i) pseudotyping and (ii) virus-like particles (VLPs). For analytical approaches solely dependent on reactivity, VLPs are a desirable reagent because of their ease of manufacture, homogeneity, and lack of safety concern (22).

Not only is the choice of immunoassay reagents critical when designing assays for public health facilities, but equally as important is the choice of platform. While traditional 96-well plate ELISA has served as a workhorse for serosurveillance efforts for decades, several immunoassay platforms emerged to make patient sample analysis faster, more sensitive, and multiplexed at both point-of-care and centralized laboratories. One such system is the Magpix® platform developed by Luminex Corporation (2). Magpix® is similar to ELISA in that it relies on a typical antigen/antibody interaction detection methodology, but employs 6 μm, fluorescently labeled magnetic particles as the solid support for the immunoassay. This allows for an increase in surface area in addition to faster assay times relative to a standard 96-well immunoassay. As a result, sensitivity is increased. By using a LED/CCD excitation and detector system, multiplexed assays are enabled by conjugating distinct assay reagents to spectrally unique bead sets (17, 19). This ability to multiplex while maintaining assay sensitivity is crucial for effective serosurveillance efforts to understand and control spread of disease.

Aside from the need for a potential field-forward immunoassay to be sensitive is the need for such an assay to be sustainable and stable (18, 23). The burden of disease is so great in many of these developing nations that the need for tests often exceeds the supply of many commercial companies, which leaves screening gaps and a lapse of care in these centralized facilities. Additionally, shipping and storage of these assays and reagents presents a unique challenge in that cold-chain shipping and freezer space/availability is often not reliable (21). This places an extreme challenge on diagnostic assay developers to design reagents and platform technologies that can operate and give reliable results under such conditions.

A need exists for sustainable, stable, and sensitive immunodiagnostics for use in public health efforts to understand and combat the threat of emerging infectious diseases. It has been discovered that it is possible to produce an immunodiagnostic reagent based on incorporation of VLPs into a magnetic bead-based immunoassay platform that is easy to produce, thermostable, and has a 2-log, or 100-fold, improved sensitivity over traditional methods.

### SUMMARY OF THE INVENTION

In one aspect, the present invention relates to a thermostable complex, comprising

(a) at least one virus-like particle (VLP) that presents at least one viral glycoprotein antigen on its surface; and

(b) a microsphere or bead that is coupled to the VLP; wherein the complex is capable of serving as an immunoassay platform for detection of an immune response.

In another aspect, the present invention relates to a method of making a thermostable complex, comprising the following steps:

(a) generating a virus-like particle (VLP) that presents at least one viral glycoprotein antigen on its surface in eukaryotic cell culture via transient expression of DNA constructs encoding structural protein(s) and antigen of interest;

(b) purifying the VLP from culture supernatant and characterizing VLP reactivity against control sera containing antibodies of interest; and

(c) conjugating the purified VLP to a microsphere substrate to generate a thermostable complex.

In yet another aspect, the present invention relates to a method for detecting an immune response to at least one antibody in a biological sample from a subject comprising:
(a) providing at least one thermostable VLP-conjugated microsphere (VCM) complex, wherein each thermostable VCM complex comprises: (i) a virus-like particle (VLP) comprising a viral glycoprotein antigen on its surface and
(ii) a detectably labeled magnetic microparticle or magnetic bead conjugated to the VLP;
(b) contacting the thermostable VCM complex with a biological sample from a subject wherein if present, an antibody from the biological sample binds to the viral glycoprotein antigen presented on the VLP of the VCM complex;
(c) determining an amount of antibodies that bind to viral glycoprotein antigens presented on the VCM complex;
(d) determining an amount of antibodies that do not bind to viral glycoprotein antigens presented on the VCM complex;
   wherein immunodetection of antibodies in the biological sample reflects viral infection in the subject and lack of immunodetection of antibodies in the biological sample reflects no viral infection.

In yet another aspect, the present invention relates to a method for detecting the presence of a target antibody in a sample, the method comprising:
(a) contacting the sample with a VLP-conjugated microsphere (VCM) complex, wherein the VCM complex comprises: (i) a virus-like particle (VLP) comprising a viral glycoprotein antigen on its surface and (ii) a detectably labeled microparticle or bead conjugated to the VLP, under conditions such that if the target antibody is present in the sample, it will bind in a detectable fashion to the thermostable VCM; and
(b) detecting whether any target antibody has bound to the VCM.

In yet another aspect, the present invention relates to a kit for detecting the presence of a target antibody in a sample, which comprises (a) a virus-like particle (VLP) comprising a viral glycoprotein antigen on its surface and a detectably labeled microparticle or bead conjugated to the VLP; (b) suitable packaging material; (c) optional control materials; and (d) optional instructions for use of the kit.

It has been discovered that it is possible to create a VCM complex as an immunodiagnostic that is easy to produce, is thermostable, and has a 2-log, or 100-fold, improved sensitivity over traditional methods. In one aspect, the disclosure provides for a VCM complex that comprises VLPs that present on their surface viral glycoprotein antigens selected from the group consisting of: alphavirus family, arenavirus family, Filovirus family, bunyavirus family, or flavivirus family. These VLPs that are approximately 100 nm in spherical diameter, were coupled or conjugated to magnetic fluorescent microspheres to create thermostable VLP-conjugated microspheres (VCMs).

In some aspects, microparticles are spherical and are about 0.1 μm to about 20 μm in diameter. Preferably, the microparticle is about 5-6 μm in diameter. These VCMs prove stable when lyophilized and stored at 37° C. and were able to detect IgG and IgM in non-human primate (NHP) and human clinical sera at dilutions of 1×10⁵ and 1×10⁴, respectively. In another aspect, when incorporated into the Magpix® platform, the VCMs were multiplexable for differential diagnosis and yielded a faster sample-to-answer time over traditional ELISA methods. This VCM complex will allow more rapid and efficient detection of known and emerging viral pathogens in human populations.

The invention further provides a VCM in a preferred embodiment, wherein the VLP portion comprises a viral glycoprotein antigen and a scaffold for antigen presentation, i.e., a retroviral core, or preferably, a MLV-Gag core. Conjugation of the VLP to the microparticle to form a VCM results in an ability to detect antibodies of an immune response. The VLP can be conjugated to the microparticle by covalent interaction or by non-covalent interaction. In preferred embodiments, the VLP presents viral glycoprotein antigens derived from a virus such as Crimean-Congo hemorrhagic fever virus (CCHF), Chikungunya virus (CHIK), Dengue virus (DENV), Eastern equine encephalitis virus (EEEV), Lassa virus (LASV), Marburg virus (MARV), Venezuelan equine encephalitis virus (VEEV), or Western equine encephalitis virus (WEEV).

In a final aspect, methods are provided for making a stable VCM and its use as a sustainable diagnostic reagent for detecting antiviral glycoprotein antibody response selected from the group consisting of: alphavirus family, arenavirus family, Filovirus family, bunyavirus family, or flavivirus family in NHP sera. First, VLPs are generated in eukaryotic cell culture via transient expression of DNA constructs encoding structural protein(s) and antigen of interest. Then, the VLPs are purified from culture supernatant and characterized against control sera containing antibodies of interest. Finally, the VLPs are conjugated to microparticle substrates to generate a VCM.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1A-1D are photographs of imaging and graphs of VEEV VLPs stained with anti-E1(A) or anti-E2 (B) mAbs. VLPs were imaged at 80,000× magnification. Scale bars are indicated for 200 nm. (C) VEEV VLPs or y-irradiated TC83 virus were coated onto ELISA plates at indicated amounts, and probed with mAbs against either VEEV EI or E2 or a negative control mouse antibody or (D) negative and VEEV-reactive sera from NHPs.

FIG. 2A-2D are graphs that represent A) stability of VEEV VCMs post-lyophilization at 4° C., RT, and 37° C. for up to 1 month, B) Limit of detection of anti-VEEV IgG in NHP sera using the VEEV VCM Magpix assay. Signal was statistically significant (p=0.01; unpaired t-test) over baseline to a dilution of 1E5, C) Comparison of VCM assay to traditional direct ELISA for detection of anti-VEEV IgG in NHP sera, D) VEEV and CHIKV multiplex assay for detection of anti-VEEV or anti-CHIKV IgG in positive NHP sera.

FIG. 3 illustrates detection of anti-VEEV IgM in Day 4 (gray) and Day 8 (black) NHP serum samples at a 1:100 dilution.

FIG. 4A-4B are bar graphs that illustrate detection of anti-VEEV IgM in VEEV challenged NHP at (A) Day 4 and (B) Day 8 time points. Four dilutions of sera were assayed in the VEEV VCM IgM assay at dilutions of 1:100, 1:500, 1:1000, and 1:5000, with this data shown for each NHP from left to right.

FIG. 5A-5B illustrate VEEV VLP entry characterization. In FIG. 4A, MLV-VEEV VLPs doped with a beta-lactamase-Gag fusion protein were mixed with either naive sera from non-vaccinated NHPs or sera from NHPs vaccinated three times with a VEEV GP plasmid and incubated at 37° C. for 1 hour. VLPs were then infected onto Vero E6 cells for 1 hour at 4° C., and incubated at 37° C. for 4 hours. VLP entry into the target cell cytoplasm was then measured by quantification of percent cells with beta-lactamase activity by flow cytometry. MLV-VEEV (FIG. 4B) VLPs were run on a SDS-PAGE reducing gel and probed with monoclonal antibodies against MLV Gag and VEEV E2. Molar ratios of plasmids used to generate VLPs are indicated.

FIG. 6 illustrates additional VEEV VCM characterization. The effect of VLP loading concentration on VCM signal with VEEV NHP sera was demonstrated.

FIG. 7A-B illustrates cross-reactivity of Old-World (CHIKV) and New-World (VEEV) alphaviruses for (A) IgG detection and (B) IgM detection from human clinical sera at a 1:100 dilution.

FIG. 8A-8B is a graph that illustrates the dynamic range for IgG and IgM detection in (A) VEEV and (B) CHIKV human clinical samples as compared to normal human samples. LoD for IgG was at a sample dilution of 1×10⁵ for both VEEV and CHIKV. LoD for IgM was at a sample dilution of 1×10³ for both. All assays were performed in triplicate. Error bars represent standard deviation.

FIG. 9 is a bar graph that illustrates IgG response to members of the filovirus (MARV), arenavirus (LASV), filovirus (EBOV), bunyavirus (CCHF), alphavirus (CHIK), flavivirus (DENV), alphavirus (VEEV), alphavirus (EEEV), and alphavirus (WEEV), using the described VCM method for each virus.

FIG. 10 depicts a VCM according to the present invention.

FIG. 11 depicts a general VCM magnetoimmunoassay for detection of both antiviral IgG and IgM responses in a serum sample.

### DETAILED DESCRIPTION OF THE INVENTION

In the Summary above, in the Detailed Description, and the claims below, as well as the accompanying figures, reference is made to particular features of the invention. It is to be understood that the disclosure of the invention in this specification includes all possible combinations of such particular features. For example, where a particular feature is disclosed in the context of a particular embodiment or embodiments of the invention, or a particular claim, that feature can also be used, to the extent possible, in combination with and/or in the context of other particular embodiments and embodiments of the invention, and in the invention generally. For the purposes of explanation, numerous specific details are set forth in order to provide a thorough understanding of the present invention. It will be apparent, however, to one skilled in the art that the present invention may be practiced without these specific details.

The invention is based on the discovery that VLPs conjugated to microparticles may be used for diagnostic purposes. VLPs coupled or conjugated to microparticles form a VCM complex that facilitates detection of an immune response in a biological sample. Such a VCM complex is easy to produce, is thermostable, and has a 2-log, or 100-fold, improved sensitivity over traditional methods. As described herein, some embodiments provide for heterologous or homologous VLPs having a retroviral core presenting viral glycoprotein antigens from viruses of the alphavirus family, arenavirus family, Filovirus family, bunyavirus family, or flavivirus family on each surface. These VCMs were shown to detect antibodies (e.g., IgG and IgM) in NHP and human clinical sera at dilutions of 1×10⁵ and 1×10⁴, respectively. These VCMs are thermostable. They remained stable at 37° C., were multiplexable, and yielded a faster sample-to-answer time over traditional ELISA methods. This VCM platform allows more rapid and efficient detection of known and emerging viral pathogens in human populations. This invention further provides compositions and methods for delivery of immunogenic molecules that offers the advantages of this VCM delivery system while also overcoming problems encountered with delivery using virus alone.

### 1. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The term “virus-like particle,” or “VLP,” as defined herein means, a non-replicating, non-infectious particle shell that contains one or more virus proteins, lacks the viral RNA and/or DNA genome, and that approximately resembles live virus in external conformation. The term “inside” a VLP when made in reference to the location of a polypeptide sequence means that the polypeptide sequence is located on the inner surface of the virus-like particle, and is encapsulated by the virus-like particle such that the polypeptide sequence is not exposed on the outside surface of the virus-like particle. Preferably, though not necessarily, the polypeptide that is inside the VLP is not accessible to binding with antibodies that are present outside the VLP. In a preferred embodiment, the VLP of the present invention comprises a viral glycoprotein antigen selected from the alphavirus family, arenavirus family, Filovirus family, bunyavirus family, or flavivirus family and a retroviral core, preferably, a MLV-Gag core.

The term “VLP-conjugated microparticle,” or “VCM,” as defined herein means, a “virus-like particle” conjugated or coupled to a microparticle. In some embodiments, the VCM comprises a magnetic microparticle that serves as the substrate for the surface bound diagnostic reagent.

The term “administering” as used herein, means a “VLP-conjugated microparticle,” or “VCM,” may be administered or performed using any of the various methods or for delivering a biologically active agent.

The terms “antigen,” “immunogen,” “antigenic,” “immunogenic,” “antigenically active,” and “immunologically active” when made in reference to a molecule, refer to any substance that is capable of inducing a specific humoral and/or cell-mediated immune response.

The term “antibody” refers to an immunoglobulin (e.g., IgG, IgM, IgA, IgE, IgD, etc.) and/or portion that contains a “variable domain” (also referred to as the “Fv region”) that specifically binds to an antigen.

The term “coupled” or “conjugated,” as used herein, refers to the joining together of elements, such as by covalent or non-covalent interactions. Examples of covalent interactions include chemical bonds; and examples of non-covalent interactions include ionic interactions, van der Waals forces, and hydrophobic and hydrophilic interactions. One example of conjugation is a virus-like particle to a microparticle.

The term, “microparticle,” as used herein, means a material comprising a wall forming material and having surface charge characteristics, size and morphology capable of serving as a substrate for VLP attachment. Microparticles may be solid or porous, have a rough or smooth surface, and may have a regular or irregular shape. Examples of microparticles include, but are not limited to, microspheres, sheets, rods and tubes.

The term “biological sample,” as used herein, means a sample derived from tissue, blood, organ, plasma, urine, feces, skin, or hair from a non-human primate or human.

The terms “subject,” “host,” and “patient,” as used herein, are used interchangeably and mean the recipient of the therapy to be practiced according to the invention. The subject can be any vertebrate, but will preferably be a mammal. If a mammal, the subject will preferably be a human, but may also be a domestic livestock, laboratory subject, or pet animal.

### 2. Embodiments

Immunodiagnostics are the standard against which many biological agent detection, identification, and diagnostic technologies are compared. Antibody-based assays continue to serve as preliminary and confirmatory diagnostic formats for many infectious and non-infectious diseases, as these assays are typically rapid, sensitive, specific, reliable, and robust. Immunodiagnostic technologies are relatively unsophisticated making them available to almost any laboratory. The assays can be divided into two general categories, antigen and antibody detection assays, which vary slightly in format but share the requirement for high quality reagents. Antigen detection assays rely heavily on agent-specific antibodies, whereas antibody detection assays rely more heavily on structurally accurate, agent-specific antigen (11, 12). Often development of sensitive and specific antibodies and antigens required for development of an immunoassay is the rate-limiting step.

Antibody-based assays or immunodiagnostics is an important component of an orthogonal diagnostic system that includes PCR-based or molecular diagnostics. In disease outbreaks, diagnostics is the first line of defense in identifying the causative agent, in treatment of disease, and eventually in the control and prevention of future outbreaks. An orthogonal diagnostic system that uses sensitive and specific molecular assays (e.g. PCR) in combination with less sensitive, but more broadly reactive immunological methods (e.g. ELISA) provides the highest confidence in a diagnostic result (1, 23-24). Performance of each assay is determined by the components used in its development. These components must be sensitive, specific, stable, robust and most importantly, sustainable (18). For immunodiagnostics, sustainability is the most problematic. Generally, anti-viral humoral responses are evaluated via direct immunoassay methods, using recombinant protein, inactivated virus, or inactivated lysates from infected cells to capture any viral antibodies in a sample. Use of whole virus is often challenging as the immunoassay must be run in high level containment. Inactivated virus can be utilized at the BSL2 level, but vital epitopes are often destroyed after inactivation protocols (10). Recombinant proteins are arguably the most sustainable, as they do not require BSL3/4 facilities for production, isolation, and use. However, soluble recombinant proteins come with substantial caveats: the need for truncation to facilitate soluble release of the recombinant protein, artificial protein structure due to the lack of a membrane anchor, and/or complete resistance to recombinant expression due to heterodimeric or complex maturation behavior (3, 13, 15-16, 26).

To circumvent these issues, an ideal BSL2 diagnostic reagent would present these viral antigens to the analyte material in the context of a viral envelope structure. In lieu of authentic virus, two primary strategies exist for presenting glycoproteins on a safe heterologous, background particle: pseudotyping and VLPs. For analytical approaches solely dependent on reactivity, VLPs are a desirable reagent because of their ease of manufacture, homogeneity, and lack of safety concern.

Not only is development of immunoassay reagents critical when designing sustainable immunodiagnostic assays, but equally as important is the choice of platform. While the traditional 96-well plate ELISA has served as a workhorse for serosurveillance efforts for decades, several immunoassay platforms have emerged to make patient sample analysis faster, more sensitive, and multiplexed at both point-of-care and centralized laboratories. In some aspects of the invention, one such system is the Magpix® developed by Luminex Corporation (Austin, Tex. USA) (2). It is similar to ELISA as it relies on detection of a typical antigen/antibody interaction, but employs 5 μm, fluorescently labeled magnetic particles as the solid support for the immunoassay, which results in faster assay times, increased sensitivity, and multiplexing capability (17,19). This ability to multiplex on an open sourced system while maintaining assay sensitivity is crucial for effective serosurveillance efforts to understand and control spread of disease.

It has been discovered that novel diagnostic reagents may be formulated by pairing a VLP with the sensitivity of magneto-immunoassay platforms to serve as a versatile tool for detection of antibodies in a biological sample. Herein, the design and implementation of a novel diagnostic reagent is disclosed, where the sustainability of a VLP is paired with the sensitivity of a platform to serve as a versatile tool for detection of antibodies in a serum sample.

In some embodiments, EEEV, VEEV, WEEV, EBOV, MARV, and LASV glycoproteins were incorporated onto a retroviral core VLP and conjugated to fluorescent, magnetic microspheres to create VCMs. The VCMs were stable to lyophilization and storage post-lyophilization at 4° C., RT, and 37° C. When incorporated into the Magpix® platform, the VCMs were shown to detect both IgG and IgM in NHP and human clinical samples with enhanced sensitivity over traditional ELISA formats, in both a singleplex and multiplex format. It has been demonstrated in certain embodiments that VCMs are viable as sustainable immunodiagnostic reagents for improved serosurveillance and public health campaigns. Furthermore, this reagent can have far reaching implications on improving diagnostic capacity at the point of care.

### A. Virus-Like Particles

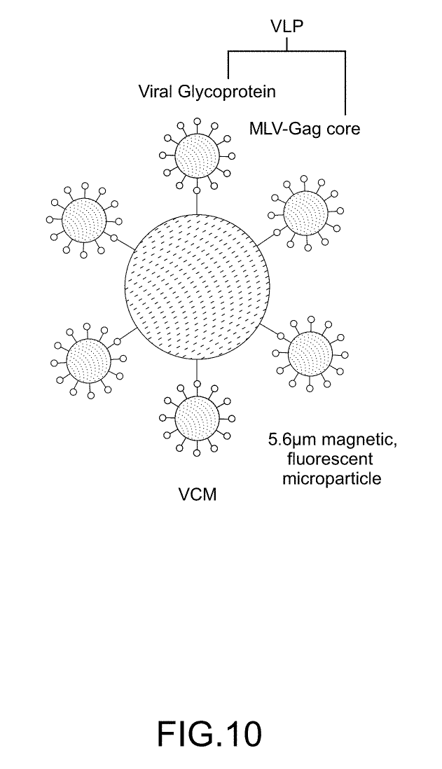

VLPs that may be used in this VCM complex are broad and encompass any variety of VLPs. In some embodiments, the VLP presents viral glycoprotein antigens selected from the group consisting of: alphavirus family, arenavirus family, Filovirus family, bunyavirus family, or flavivirus family. In other embodiments, the VLP presents viral glycoprotein antigens selected from the group consisting of: Crimean-Congo hemorrhagic fever virus (CCHF), Chikungunya virus (CHIK), Dengue virus (DENV), Eastern equine encephalitis virus (EEEV), Lassa virus (LASV), Marburg virus (MARV), Venezuelan equine encephalitis virus (VEEV), or Western equine encephalitis virus (WEEV). As shown in FIG. 10, at least one VLP has a retroviral core (e.g., a MLV-Gag core) and presents at least one viral glycoprotein antigen on its surface; and a microsphere or bead that is coupled to the VLP. In some embodiments, the VLP is a homologous VLP or a heterologous VLP VLPS are from 20-200 nm in diameter.

### B. Microparticles

Microparticle morphology can include spheres, sheets, rods, tubes and other shapes, and be solid or porous. The microparticles can have smooth surfaces, angular surfaces, rough surfaces, porous surfaces, or sharp edges. Microparticle size can vary over a fairly broad range, e.g., from about 0.1 μm to about 40 μm in diameter or length, and still be effective. In one embodiment, the microparticles are about 0.5 μm to about 20 μm in diameter or length. Preferably, the microparticle diameter or length is about 1 to about 10 or about 5-6 μm.

Microparticle materials can comprise any of a wide range of particles, including such exemplary wall forming materials as described in U.S. Pat. No. 5,407,609. Biocompatible materials are preferred for uses that involve administration to patients. Biodegradable materials are also preferred. Preferred are biodegradable polymers, such as poly(lacto-co-glycolide) poly(lactide), poly(glycolide), poly(caprolactone), poly(hydroxybutyrate) and/or copolymers thereof. Alternatively, the microparticles can comprise another wall-forming material. Suitable wall-forming materials include, but are not limited to, poly(dienes) such as poly(butadiene) and the like; poly(alkenes) such as polyethylene, polypropylene, and the like:, poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. These materials may be used alone, as physical mixtures (blends), or as copolymers.

Biodegradable microspheres (e.g., polylactate polyglycolate) for use as carriers are disclosed, for example, in U.S. Pat. Nos. 4,897,268; 5,075,109; 5,928,647; 5,811,128; 5,820,883; 5,853,763; 5,814,344; 5,407,609; and 5,942,252; the disclosures of each of which are incorporated herein by reference. In particular, these patents, such as U.S. Pat. Nos. 4,897,268 and 5,407,609, describe the production of biodegradable microspheres for a variety of uses, but do not teach the optimization of microsphere formulation and characteristics for DNA delivery.

Magnetically responsive microparticles of the present invention are prepared by heterocoagulating colloidally stable aqueous dispersions of magnetically responsive material including ferrofluids, super ferrofluids, etc., such as paramagnetic or, preferably, superparamagnetic magnetite onto the surface of core particles. The cores and the magnetite are oppositely charged and heterocoagulate initially due to electrostatic attraction, and heterocoagulate further upon the addition of heterocoagulant. After the desired degree of hetercoagulation has been accomplished a polymeric dispersant is added which disperses the heterocoagulated magnetite-coated microparticles so as to suspend them in solution where they remain in Brownian motion in the absence of a magnetic field. If desired, the dispersed magnetite-coated microparticles may be crosslinked and/or further coated with one or more outer polymeric coatings.

Microparticles labeled with fluorescent dyes have found use in a wide variety of applications. Fluorescent microparticles are most commonly used in applications that can benefit from use of monodisperse, chemically inert particles that emit detectable fluorescence and that can bind to a particular substance in the environment. The high surface area of microparticles provides an excellent matrix for attaching molecules that selectively bind to targets, while the fluorescent properties of these particles enable them to be detected with high sensitivity. They can be quantitated by their fluorescence either in aqueous suspension or when captured on membranes. Many luminescent compounds are known in the art and have proven suitable for imparting bright and visually attractive colors to various cast or molded plastics such as polystyrene and polymethyl methacrylate. Uniform fluorescent latex microspheres have been described in patents (U.S. Pat. No. 2,994,697, 1961; U.S. Pat. No. 3,096,333, 1963; Brit. Patent 1,434,743, 1976) and in research literature (Molday, et al., J. CELL BIOL. 64, 75 (1975); Margel, et al., J. Cell Sci. 56, 157 (1982)). Brinkley, et al., Ser. No. 07/629,466, filed Dec. 18, 1990 describes derivatives of the dipyrrometheneboron difluoride family of compounds (derivatives of 4,4-difluoro-4-bora-3a,4a-diaza-s-indacene) as useful dyes for preparing fluorescent microparticles. This family of dyes possesses advantageous spectral data and other properties that result in superior fluorescent microparticles.

Microparticles can be conjugated to the VLP by covalent interaction or by non-covalent interaction. Examples of covalent interactions include chemical bonds; and examples of non-covalent interactions include ionic interactions, van der Waals forces, and hydrophobic and hydrophilic interactions.

### C. Method of Making a ThermoStable VCM as a Sustainable Diagnostic Reagent

Methods are provided for making a thermostable VCM. First, VLPs are generated in eukaryotic cell culture via transient expression of DNA constructs encoding structural protein(s) and antigen of interest. Then, the VLPs are purified from culture supernatant and characterized against control sera containing antibodies of interest. Finally, the VLPs are conjugated to microparticle substrates to generate a VCM.

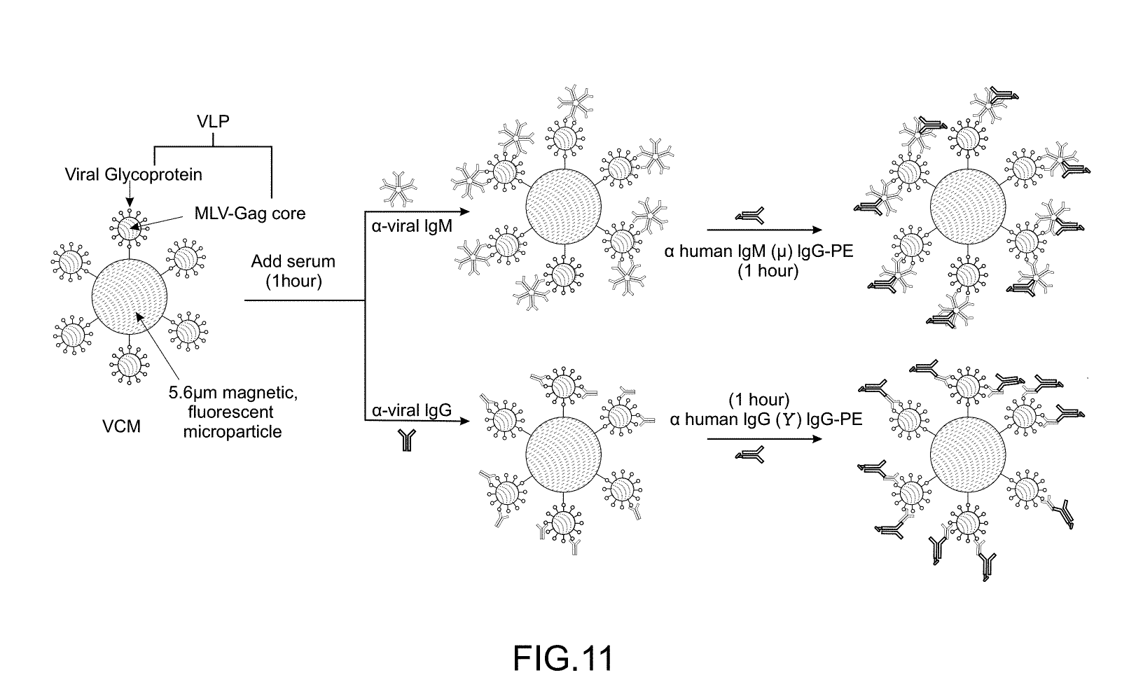

In one embodiment, viral glycoprotein antigens (e.g., VEEV E1/E2 glycoproteins) were incorporated onto a retroviral core VLP as described herein. To date, the E1/E2 heterodimer of VEEV has not been successfully purified in an intact recombinant form (27), necessitating an alternate platform for antigen presentation. After characterization, the VEEV VLPs were conjugated to fluorescent, magnetic microparticles to create VLP-coupled microparticles (VCMs). These VCMs were shown to detect both IgG and IgM in non-human primate (NHP) and clinical human serum samples, in two hours, with 2-log, or 100-fold, greater sensitivity over traditional cell lysate-based direct ELI SA as shown in FIG. 11.

The VCMs were shown to be stable to lyophilization and storage post-lyophilization at 4° C., RT, and 37° C. In other embodiments, the VCM platform was used with another viral pathogen, CHIKV, to create a multiplex with similar assay sensitivities as singleplex VEEV and CHIKV assays. The invention demonstrates how VCMs are viable as sustainable reagents for improved serosurveillance and public health campaigns. Therefore, such can have far reaching implications on improving the quality and time to result generated at the point of care.

The VCM is comprised of sustainable reagents. Many traditional immunoassays utilize inactivated viral preps or recombinant antigens as capture reagents in a direct assay format; however these reagents can be costly to produce, lack structural fidelity to native particle-associated antigen, and in the case of viral preps, require higher level containment (BSL3/4) for production of virus prior to inactivation. Therefore, in an embodiment, using a retroviral core-based VLP as a capture antigen is highly advantageous in that these particles are easy and inexpensive to produce, safe to use as they are comprised of non-infectious material, and maintain native and structural conformation of the surface antigen of interest.

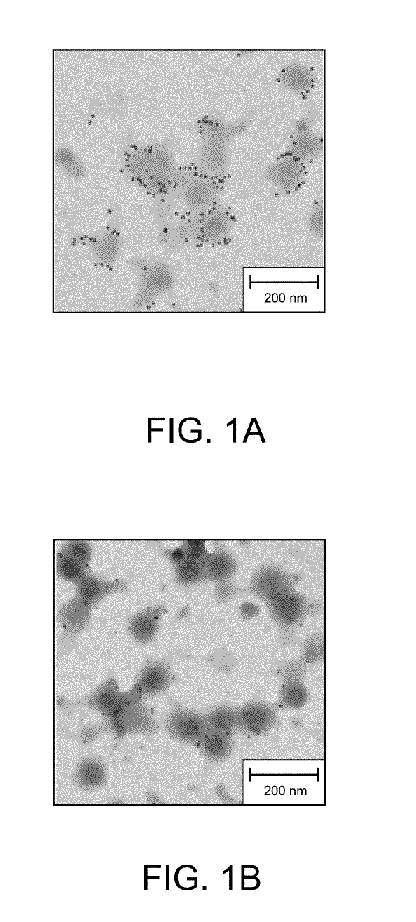

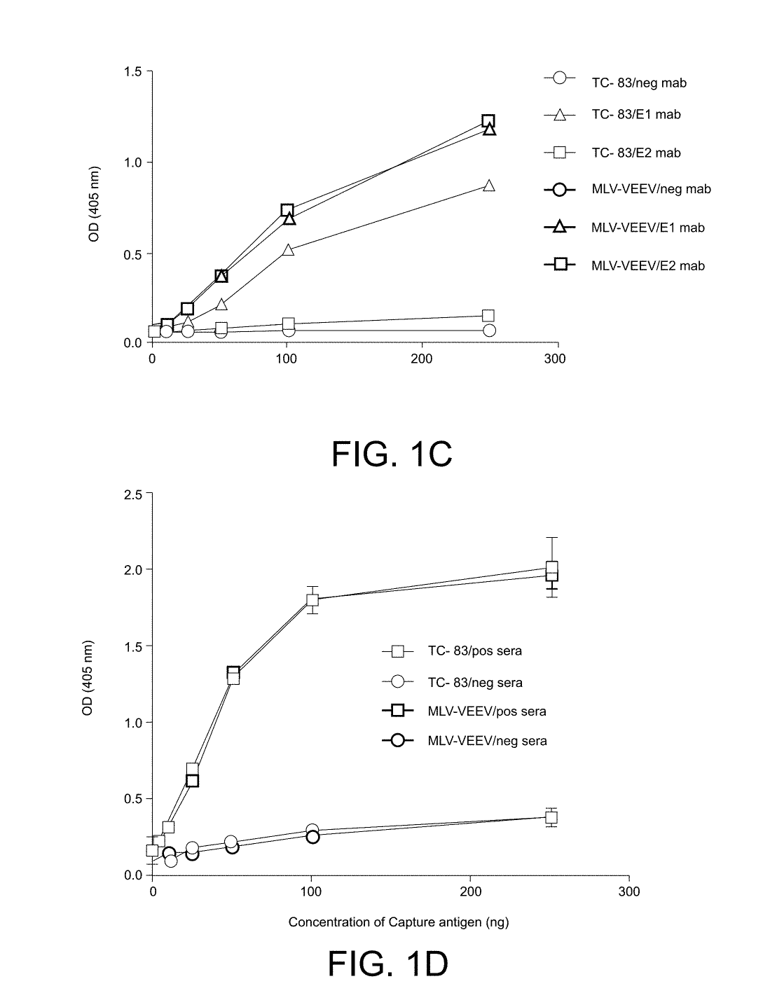

To facilitate detection of antibodies against these agents, the VEEV E1/E2 were integrated onto a MLV-Gag retroviral core. The VEEV glycoprotein heterodimer is difficult to produce in a recombinant form. In some embodiments, the integration of these glycoproteins onto a stable, MLV Gag-based particle core was a rational approach for creating diagnostically useful, membrane stabilized glycoprotein targets for an immunoassay format. These particles were shown to be highly homogenous and reactive against seropositive NHP sera and glycoprotein-specific monoclonal antibodies (FIG. 1).

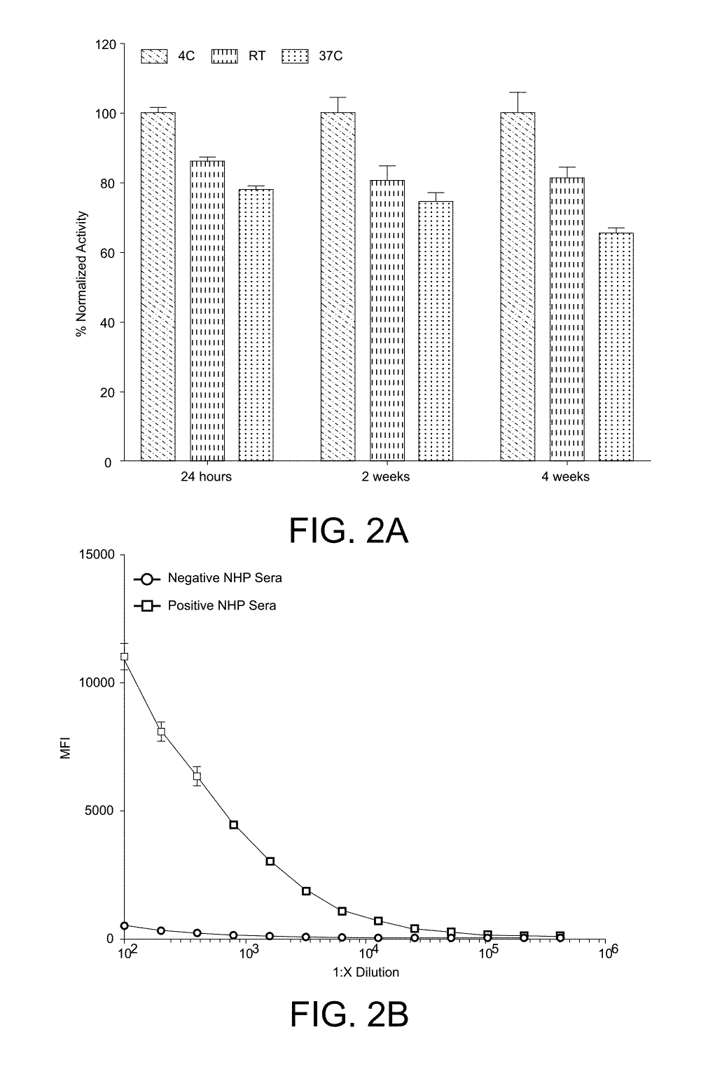

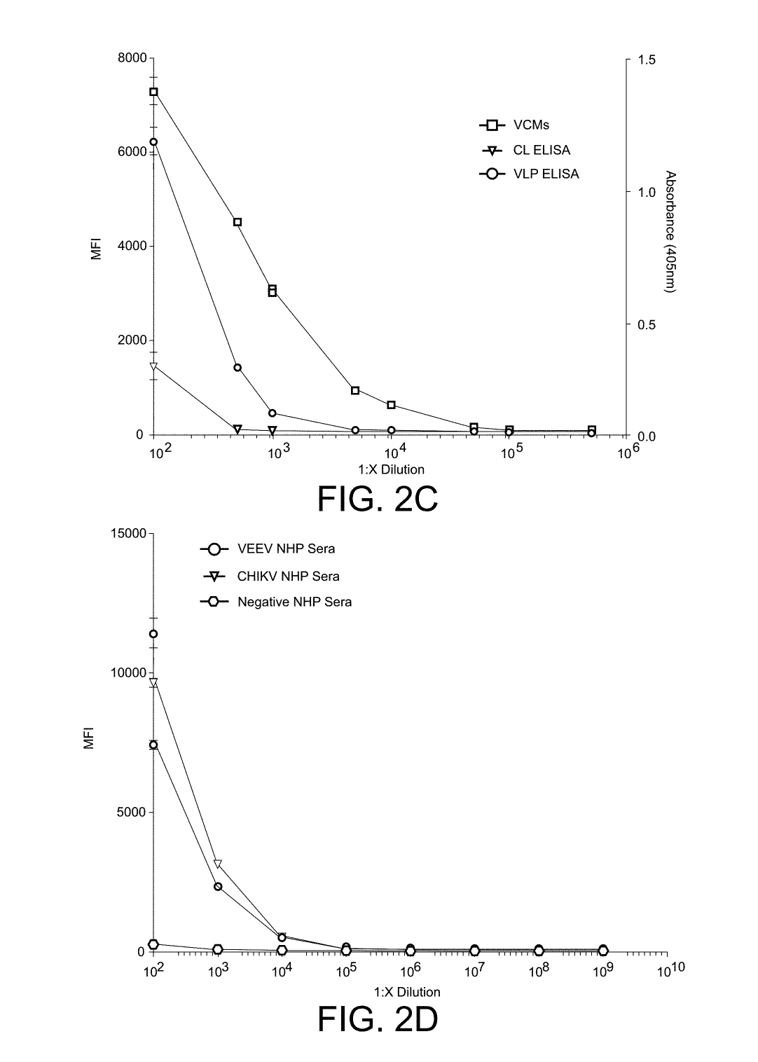

After characterizing their structural properties, in some embodiments, the VLPs were conjugated to magnetic microparticles to determine their reactivity in a microbead-based immunodiagnostic platform. Post-conjugation, the VCMs were shown to detect anti-viral antibodies in NHP sera to dilutions of 1×10⁵ (FIG. 2B-D).

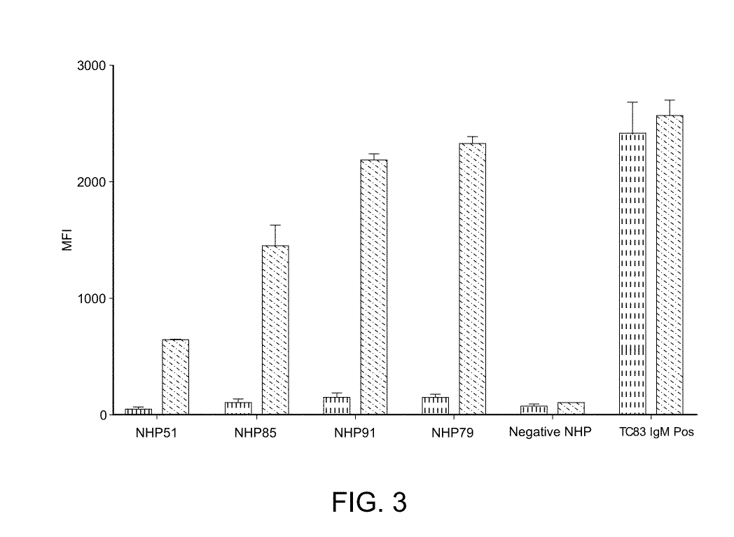

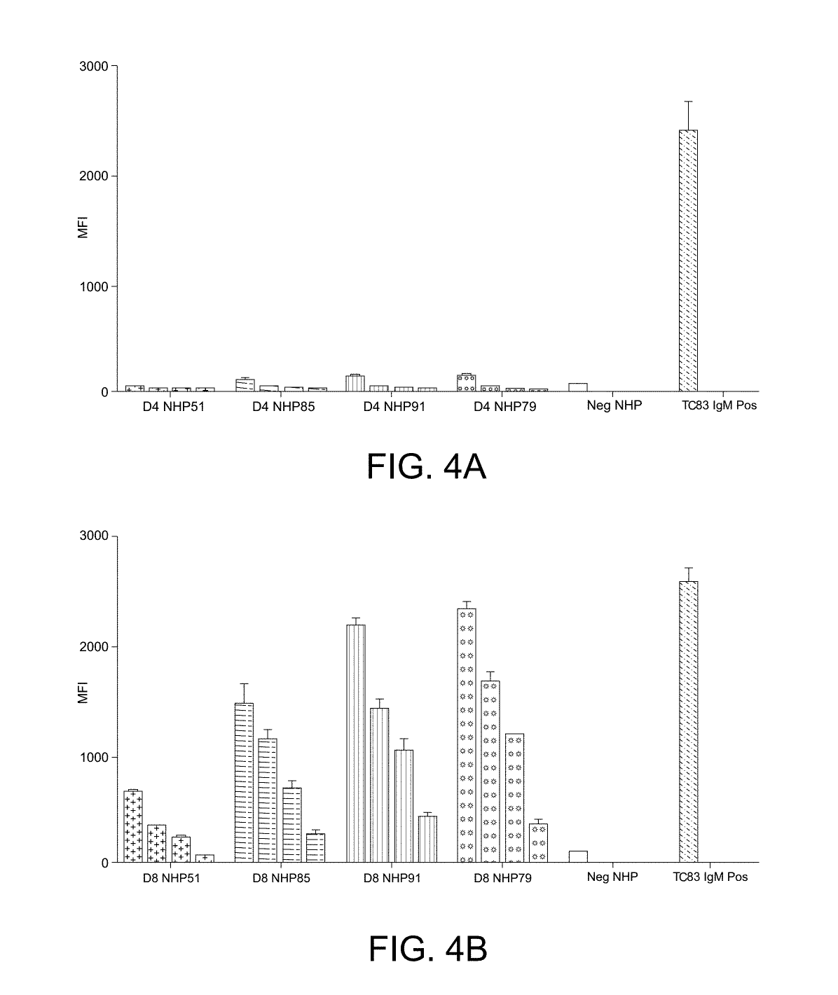

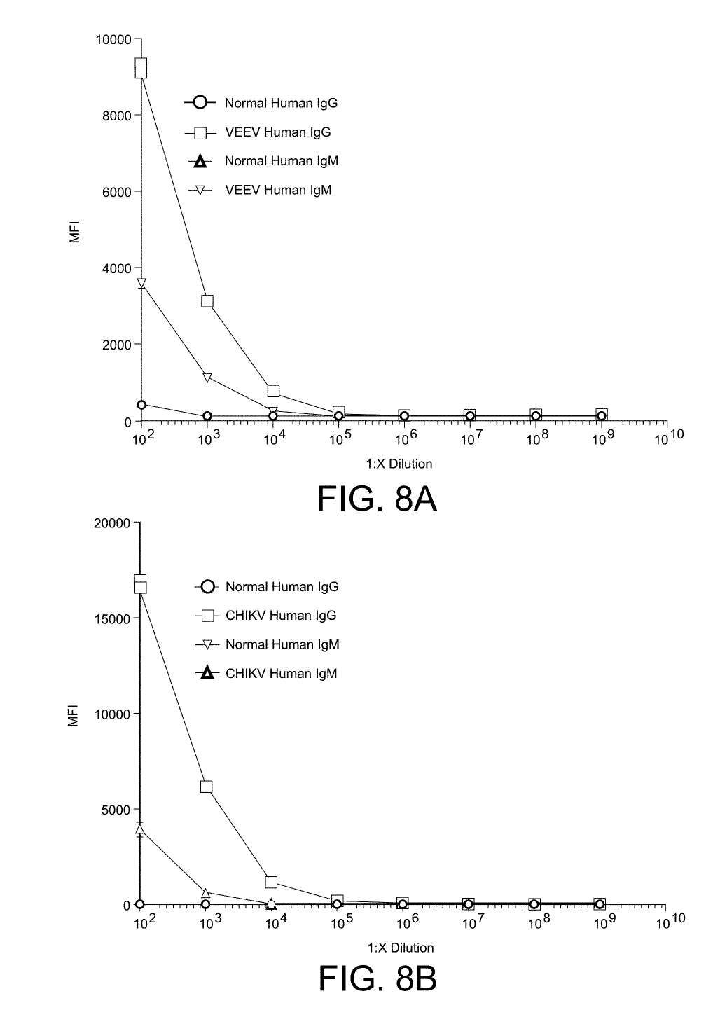

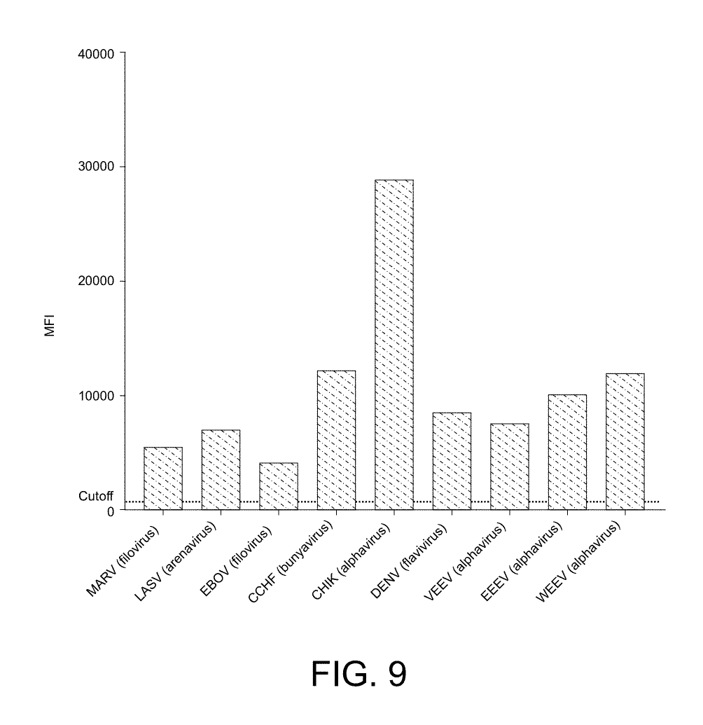

A second, important aspect of viral immunodiagnostics is stability of the reagents, as the use-scenario often involves more extreme storage conditions (i.e. heat, humidity, power surges, lack of cold chain shipping). To test stability, the VCM complex was lyophilized and shown to be stable at temperatures up to 37° C. and retain significant activity for at least 1 month (FIG. 2A). Further time points will be evaluated in the future to ensure the VCMs remain stable up to one year. Lastly, an immunodiagnostic detection platform for emerging viral pathogens should be sensitive and multiplexable enough to increase the probability of identifying populations at risk of exposure or effectively screening a population for a history of exposure. The VCM complex described herein was shown to be extremely sensitive for detection of humoral response to infection in convalescent and early time point sera. In some aspects, versatility of the particles for both IgG and IgM detection was demonstrated in both animal model samples (FIGS. 3A and 4A-B) as well as human clinical samples (FIG. 8A), where sensitivities were observed at serum dilutions of 1×10⁵ and 1×10⁴ for IgG and IgM, respectively.

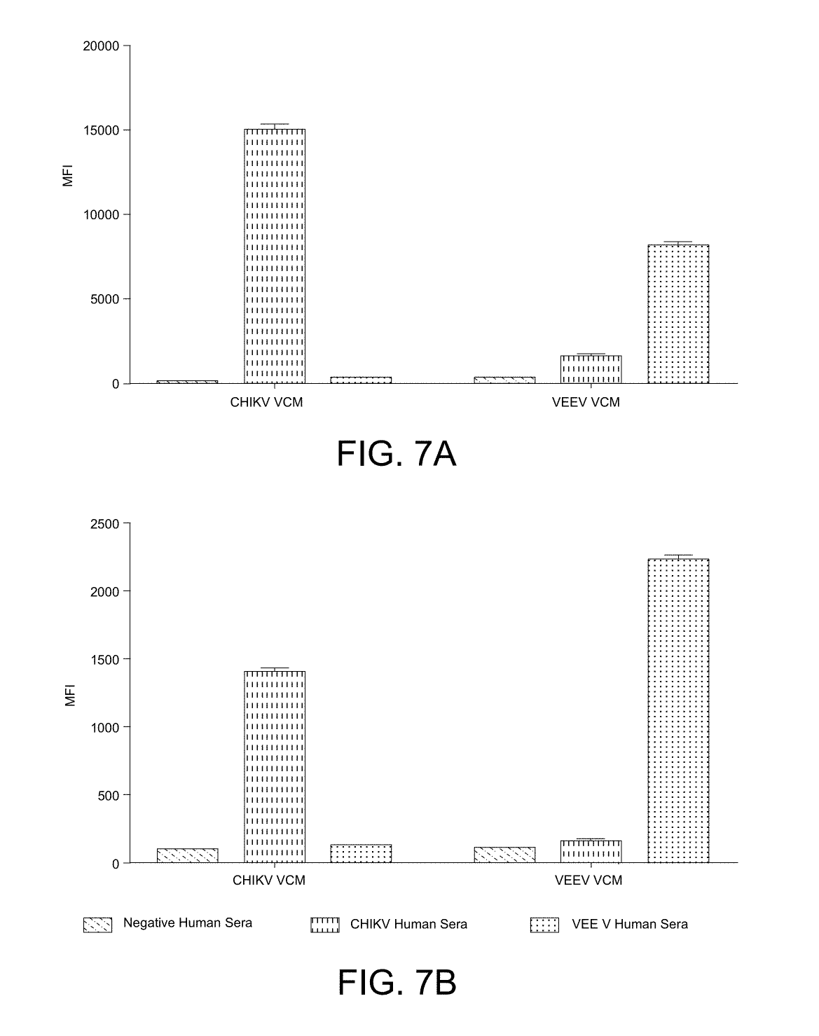

In some embodiments, the system was also shown to multiplex well for VEEV and CHIKV anti-viral antibody detection, with no observed loss in sensitivity upon the addition of additional assay components (FIGS. 2D and 7A-B). A marked improvement over traditional ELISA methods was observed, not only in a reduction in sample to answer time, but also in the two order of magnitude higher level of sensitivity (FIG. 2C). While many of these sensitivity advantages owe themselves in part to incorporation of the VCM complex in a multiplexed, magnetic bead based assay platform such as the Magpix®, the use of VLPs in such bead-based systems allows for a widened immunoassay design space, to include additional glycoprotein targets that have previously not been amenable to recombinant expression. Many overseas centralized laboratories are equipped with this instrument; however the absence or lack of access to assay content for the platform has limited their wide-spread use for public health efforts.

Without being bound by theory, it is possible to extend this VCM complex technology to include detection capabilities for viruses of the alphavirus family, arenavirus family, filovirus family, bunyavirus family, or flavivirus family, particularly those endemic in central and western sub-Saharan Africa. Furthermore, it is possible to create country-specific serosurveillance panels for use at centralized testing facilities. In addition, as many vaccine monitoring regimens are focused on monitoring serological response to glycoprotein antigens, it is anticipated that the VCM approach will be a valuable tool for both pre- and post-vaccination monitoring campaigns. As disease surveillance moves forward, both in developed and developing nations, incorporation of sustainable and sensitive reagents into field-forward technologies will become increasingly important in global efforts to limit the spread of infectious viral diseases.

### D. VCM Complex Compositions

The invention provides in other aspects, compositions that are useful for detecting antiviral IgG and IgM responses in biological sample. In one embodiment, the composition is a VCM complex that comprises an antigenically relevant VLP-bound glycoprotein antigen that can detect an immune response (i.e., IgG and IgM antibodies). In some embodiments, the condition to be detected is an infectious disease. Examples of infectious disease include, but are not limited to, infection with a pathogen, virus, bacterium, fungus or parasite. Examples of viruses include, but are not limited to, Crimean-Congo hemorrhagic fever virus (CCHF), Chikungunya virus (CHIK), Dengue virus (DENV), Eastern equine encephalitis virus (EEEV), Lassa virus (LASV), Marburg virus (MARV), Venezuelan equine encephalitis virus (VEEV), or Western equine encephalitis virus (WEEV).

The present invention is also directed to kits useful in detecting the presence of a target antibody in a sample. The kit comprises a virus-like particle (VLP) comprising a viral glycoprotein antigen on its surface and a detectably labeled microparticle or bead conjugated to the VLP. It may also contain various materials conventionally used in such kits, such as control materials, and other reagents. The kits will include suitable conventional packaging materials, as well as optional instructions for use of the kit.

### 3. EXAMPLES

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### DNA Constructs and Cells

Plasmids encoding the glycoprotein coding regions for VEEV E1/E2 (pWRG7077-coVEEV26S) have been previously described (4, 7). For the construction of the Gag-encoding plasmid, the first 538 residues of murine leukemia virus (MLV) Gag-Pol ORF (GenBank: AF033811.1) was codon optimized, synthesized, and cloned into the pWRG7077 expression vector using flanking 5′ Notl and 3′ BglII restriction sites relative to the transgene insert.

HEK293T (ATCC) cells were cultured in DMEM (Corning) supplemented with 10% heat-inactivated Fetal Bovine Serum (Gibco), 1% Penicillin/Streptomycin (Gibco), 1% L-glutamine (Hyclone), and 1% Sodium pyruvate (Sigma).

### Example 2

### Antibodies and Sera

For ELISA and Immuno-electron microscopy (IEM) analysis of VEEV VLPs, monoclonal antibodies against VEEV envelope glycoproteins E1 (3B2A9) (18) or E2 (MAB 8767, Millipore) were used. A monoclonal antibody against VSV-G glycoprotein, (1E9F9) 114, was used as a negative control. Positive and negative control sera from non-human primates (NHPs) vaccinated with either VEEV E1/E2 were generated as described previously (4, 7).

### Example 3

### VLP Production

HEK293T cells were seeded in 10 cm² round plates and incubated at 37° C. with 5% CO2 until reaching 70-80% confluency, prior to transfection with a 3:1 ratio of 9 μg WRG7077-Gag and either 3 μg pWRG7077-coVEEV26S using Fugene 6 (Roche, Indianapolis, Ind., USA) according to manufacturer's instructions. Cell supernatants were collected at 24 and 48 hr post-transfection, pooled, and clarified by centrifugation. LASV VLPs, but not VEEV VLPs, were concentrated through a Centricon filter with a 100-kDa cutoff (EMO Millipore, Billerica, Mass., USA) according to manufacturer's instructions. All VLPs were pelleted through a 20% sucrose cushion in virus resuspension buffer (VRB; 130 mM NaCl, 20 mM HEPES, pH 7.4) by centrifugation for 2 hr at 106,750×g in an SW32 rotor at 4° C. VLP pellets were re-suspended overnight in VRB at 4° C., then pooled and ten-fold diluted with VRB. The diluted VLPs were re-pelleted without a sucrose cushion as described above. VLPs were re-suspended in 1/1000 volume of VRB relative to starting supernatant, and then stored at −80° C. Protein concentration was determined by BCA assay (ThermoFisher, Waltham, Mass., USA).

### Example 4

### Conjugation of VLPs to MagPlex® Microspheres

VEEV VLPs were conjugated to MagPlex® microspheres following the protocol outlined by the Luminex xMAP® antibody coupling kit (Cat #40-50016). Briefly, 100 μL of MagPlex® beads (Bead Region #75; 12.5E6 beads/ml; Cat #MC10075-YY) were washed three times, using a magnetic microcentrifuge tube holder, and resuspended with 480 μL of the supplied activation buffer. To these resuspended microspheres, 10 μL of both sulfo-NHS and EDC solutions were added. The tube was covered with aluminum foil and placed on a benchtop rotating mixer for 20 minutes. After surface activation with EDC, the microspheres were washed three times with activation buffer prior to adding the VEEV VLPs at a final concentration of 10 μg VLPs/1×10⁶ microspheres. The tube was again covered with aluminum foil and placed on a benchtop rotating mixer for 2hours. After this coupling step, the beads were washed three times with the supplied wash buffer and re-suspended in 100 μL of wash buffer for further use. Other demonstrated VLPs were coupled to MagPlex® microspheres in a similar manner.

### Example 5

### Detection of Anti-VEEV IgG or IgM in NHP or Human Sera using VEEV VLP-Coupled MagPlex® Microspheres

VLP-bound MagPlex® microspheres were diluted in phosphate buffer saline (PBS) with 0.02% Tween-20 (PBST) to 5×10⁴ microspheres/mL and added to the wells of a Costar white, polystyrene 96 well plate (Corning Cat #3789A), at 50 μL per well (2500 microspheres/well). The plate was placed on a Luminex plate magnet (Cat #CN-0269-01), covered with foil, and allowed microspheres to collect for 60 sec. While still attached to the magnet, the buffer was removed from the plate by shaking. To appropriate wells, 50 μL of the diluted serum was added, the plate covered, and incubated with shaking, for 1 hour at room temperature (RT). The plate was washed three times with 100 μL of PBST, using the plate magnet to retain the MagPlex® microspheres in the wells, then, 50 μL of a 1:100 dilution of goat anti-human IgG (H&L) phycoerythrin conjugate (Sigma Cat #P9170), or goat anti-human IgG (H&L) phycoerythrin conjugate (Sigma Cat #P9170) or goat anti-human IgM (anti-mu) phycoerythrin conjugate (Abcam Cat #ab99739) in PBST, was added to the wells. The plate was covered and incubated with shaking for 1 hr at RT. After incubation, the plate was washed three times and the MagPlex® microspheres resuspended in 100 μL of PBST for analysis on the Magpix®. In the case of sera from viremic, VEEV-infected NHPs, analysis was conducted in a BSL-3 suite, with infected material handled in a class II Biological Safety Cabinet.

### Example 6

### Synthesis and Characterization of VEEV VLPs

A MLV-based VLP was chosen as the backbone for VEEV E1/E2 glycoprotein expression because they are high yielding, homogenous, and can accommodate a wide range of glycoprotein antigens (21, 22). Transient expression of the DNA construct containing the first 538 amino acids of MLV Gag in mammalian cells generated highly homogenous particles presenting both the E1 and E2 VEEV glycoproteins on their surface (FIG. 1A). A molar ratio of 3:1 Gag structural protein to VEEV E1/E2 glycoprotein yielded the highest incorporation of the glycoproteins into the particles (FIG. 4B). This same ratio was optimal for LASV GPC (FIG. 4C). Comparison of the EEEV E1/E2 VLPs was made against y-irradiated whole TC-83 VEEV antigen by direct ELISA. Plates coated with equal amounts of each antigen were probed with either E1 or E2 specific monoclonal antibodies (mAbs) or with sera from NHPs vaccinated with a VEEV E1/E2 DNA vaccine. The VLPs performed better as compared to the inactivated material, with respect to the mAbs and displayed equivalent reactivity in response to polyclonal sera from vaccinated NHPs. As further proof that the MLV-based VLP VEEV glycoproteins were present in a native, functional conformation, we demonstrated successful entry of the VLPs into target cells. This entry was also blocked by neutralizing polyclonal sera from vaccinated NHPs, further supporting the native-like structure of the VLP embedded glycoproteins (FIG. 4A).

### Example 7

### Conjugation, Characterization, and Lyophilization of VEEV VCMs

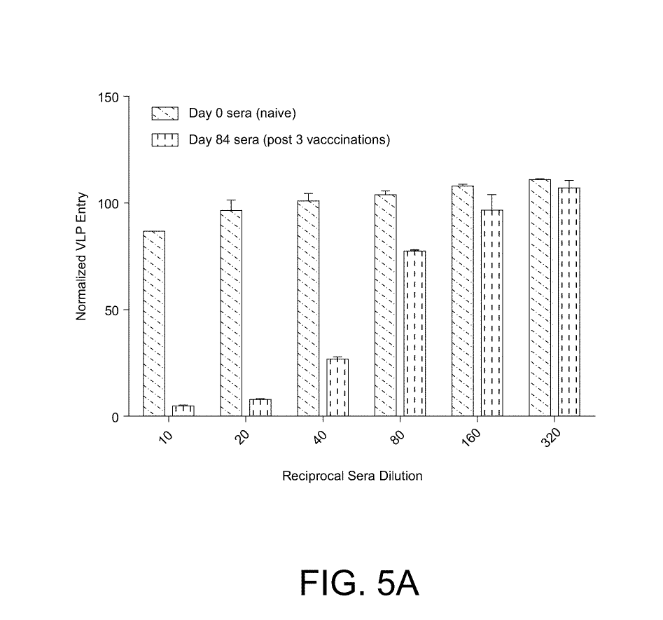

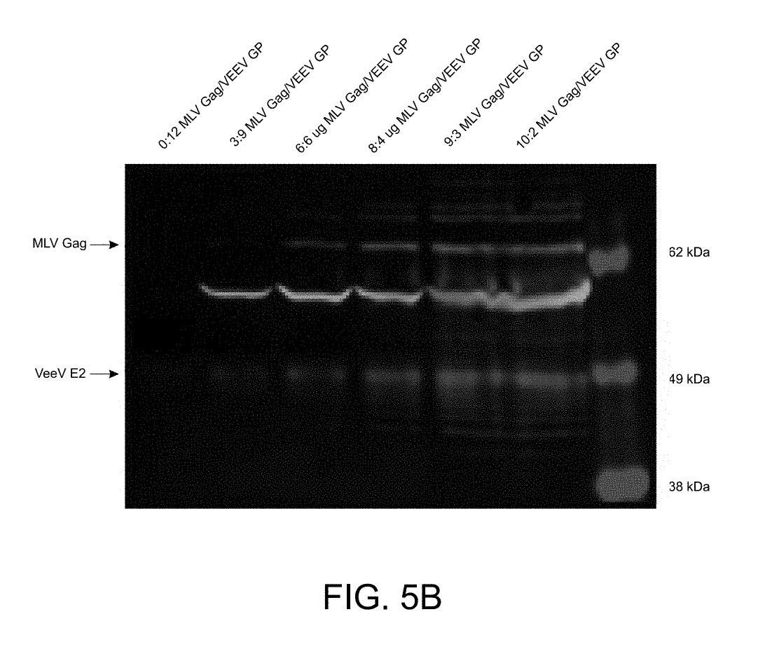

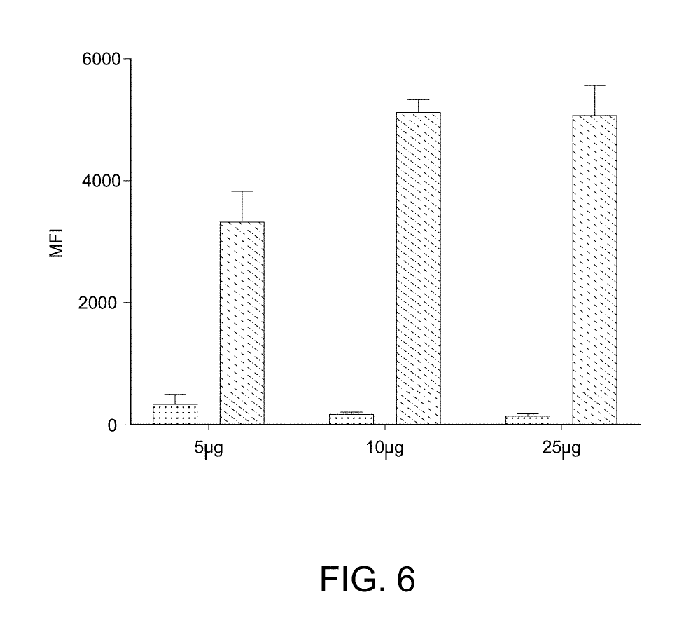

The VLPs were conjugated to MagPlex® microspheres using carbodiimide coupling chemistry to covalently link the amine groups from the surface glycoproteins of the VLP to the carboxylate surface of the microparticle. Screening VEEV infected NHP sera on the Magpix® with these VCMs yielded a signal to noise value of 12.0, which was significant compared to signal from a negative NHP serum sample (p<0.0001) (FIG. 5A). This observation was similar to that seen from the VLPs alone, indicating that the conformational integrity of the VLPs was not altered when coupled to the microspheres (FIG. 4A). Maintenance of cold-chain is an ever present requirement in austere environments; therefore we investigated the effects of lyophilization to improve stability of the VCMs. Assay components, like the VCMs, that could be stored for long periods of time without the need for cold-chain and little loss in reactivity, would be of significant benefit. VCMs and unconjugated VLPs, were resuspended in lyophilization buffer, containing stabilizers, and lyophilized overnight. For both the VCMs and the VLPs alone, 100% activity was recovered post-lyophilization (FIG. 5A). Lyophilized VCMs at 4° C., RT, or 37° C. were stored for up to 4 weeks with only minor loss of activity (FIG. 2B). An initial drop in signal was observed with the lyophilized VCMs stored at RT and 37° C. after 24 hrs, to 86% and 78%, respectively when compared to particles stored at 4° C. This positive signal was still significant over background (p<0.0001), indicating VLP reactivity was maintained at elevated storage temperatures. After 4 weeks of storage, activity of the VCMs at RT and 37° C. remained at 81% and 67%, respectively.

### Example 8

### Sensitivity of VEEV VCMs as a Singleplex and Multiplexed with CHIKV VCMs

The limit of detection (LoD) of the VCM direct assay detecting VEEV IgG positive NHP serum was determined to be a 1×10⁵ dilution in assay buffer (FIG. 2B). Signal at this dilution was statistically significant when compared to the same dilution of negative NHP sera (p<0.0001). The VEEV VCM Magpix® assay was found to be two orders of magnitude more sensitive than traditional ELISA assays using inactivated TC-83 cell lysate or VEEV VLP direct capture antigens. To determine whether this VCM reagent could also be used in a multiplexed format, CHIKV VLPs were synthesized, conjugated to MagPlex® microspheres, and optimized with CHIKV IgG positive NHP sera. The limit of detection of the multiplex assays, detecting either VEEV or CHIKV IgG positive NHP sera, were each at a 1×10⁵ dilution of positive serum, which was identical to the LoDs of the individual singleplex assays.

### Example 9

### Diagnostic Utility of VEEV VCMs for Animal Models and Human Clinical Samples

While the VCMs proved to be highly sensitive for detection of IgG in convalescent NHP sera, the diagnostic utility of such a platform lies in its sensitivity toward both IgM and IgG detection in early time point sera from both animal models and human clinical samples. The presence of IgM is the earliest antibody indicator of infection the body makes against a pathogen. As the course of infection progresses toward convalescence, the presence of IgM decreases as IgG rises to dominate the humoral response (24). To test the VEEV VCM assay for detection of IgM in NHP serum samples, day 4 and day 8 (post-infection) serum samples from four VEEV infected NHPs were screened at a 1:100 dilution (FIG. 3A). Anti-VEEV IgM response could be detected at day 4 post-challenge in three out of four NHPs. IgM response increased dramatically between days 4 and 8 and was detectable at a dilution of 1:5000 (FIG. 4A-B).

Additionally, human clinical samples of known VEEV and CHIKV infection were screened for the presence of anti-IgG and IgM antibodies (FIG. 8A-B). Similar to the convalescent NHP serum samples discussed previously, the VEEV IgG VCM assay could detect antibody at a 1×10⁵ dilution (FIG. 8A). For human serum samples known to be IgM positive, the assay could detect antibody at a dilution of 1×10⁴ for anti-VEEV IgM detection (FIG. 8A). Acute and convalescent human sera, of known VEEV and CHIKV etiology, were screened with VEEV and CHIKV VCMs to demonstrate IgM and IgG detection in human clinical samples (FIG. 7A-B). When screened in a VEEV/CHIKV duplex, no crossreactivity was observed for IgM detection, with minimal crossreactivity for IgG detection.

### Example 10

### VLP Characterization

For western blot detection, VLPs were run under reducing conditions on an SDS-PAGE gel, transferred to nitrocellulose, blocked with Odyssey Blocking Buffer (Licor), and probed with rabbit anti-MLV Gag polyclonal antibody (Abcam) and mouse anti-VEEV E2, clone 1A4A-1 (18) at working concentrations of 0.4 μg/ml and 1 μg/ml respectively. Blots were developed by probing the membrane with 1:10,000 dilutions of anti-mouse IR680 (Licor) or anti-rabbit IR800 (Licor).

For immuno-electron microscopy (IEM) analysis of VLPs, MLV-VEEV VLPs were adsorbed to formvar/carbon coated nickel grids. Grids then were incubated with 1:500 dilutions of mAbs against either VEEV envelope glycoproteins E1 (3B2A9)[1] or E2 MAB 8767 (Millipore), respectively. Labeled samples were then probed with a 1:500 dilution of anti-mouse IgG conjugated with 100 nm immuno-gold particles. After immuno-staining, grids were then negative stained with 1% PTA for contrast. Samples were evaluated on a JEOL 1011 transmission electron microscope at 80 kV and digital images were acquired using AMT camera system.

### Example 11

### VLP Entry Fitness

MLV-VEEV VLPs were produced as described with the following changes. In addition to the VEEV E1/E2 coding plasmid, the DNA transfection mixture contained a 1:1 mixture of MLV-Gag and MLV-Gag with a Beta-lactamase reporter fused to the N-terminus of Gag. VLP supernatants were harvested at 24 and 48 hours prior to being clarified and pelleted through 20% sucrose, resuspended in VRB and frozen for later use. One day prior to the entry assay, target Vero E6 cells were seeded the day before in 96-well plates at a density of 50,000 cells per well. VLPs diluted in media were then incubated for 1 hour at 37° C. with the indicated dilutions of sera from naive (non-vaccinated) NHPs or with sera collected after 1 (day 28) or three vaccinations (day 84) with a VEEV E1/E2 encoding plasmid. VLP/sera mixtures were then spinfected onto cells at 250×g rpm for 1 hr at 4° C. Cells were then incubated at 37° C/5%CO2 for 4 hours to permit VLP entry. Cells were processed for detection of beta-lactamase entry signal as previously described (26). Beta lactamase cells were quantified on a FACS Canto (BD) flow cytometer, and were reported as percentage of positive cells relative to cells loaded with only ccf2-am substrate.

### Example 12

### Direct ELISA

High-bind ELISA plates (Thermo Fisher Cat #3455) were coated with the indicated amounts of either 1 μg/mL MLV-VEEV VLP or a 1:1000 dilution of irradiated TCS3 capture antigen diluted in PBS and incubated overnight at 4° C. The plate was washed three times with PBST using a Biotek 405TS automatic plate washer. After washing, 250 μL of 5% skim milk buffer was added to the wells to block the well surface against nonspecific interactions. The plate containing the blocking buffer was placed in a 37° C. incubator for 1 hour. After blocking, the plate was washed with the plate washer before adding 100 μL of the negative and positive NHP serum dilutions to their respective wells. The plate was again placed in in a 37° C. incubator for 1 hour. After 1 hour, the plate was washed before adding 100 μL of a 1:10000 dilution of goat-antihuman IgG peroxidase labeled secondary (KPL Cat #074-1006) to each well and incubated for 1 hour at 37° C. Following the conjugate step, the plate was washed before adding ABTS developer (KPL Cat #5120-0032) for 1 hour at 37° C. After one hour, the plate was read using a Tecan Infinite® 200 PRO microplate reader at 405 nm, with a reference reading at 490 nm.

### Example 13

### Lyophilization of VLP conjugated MagPlex® Microspheres

VEEV VLP conjugated MagPlex® microspheres were diluted in lyophilization buffer (40% mannitol, 0.5% Tween-20 in PBS; Chuan 2012) to a 500,000 beads/ml concentration. 100 μL of the diluted microspheres were aliquoted into 2 mL microcentrifuge tubes. The caps of the tubes were punctured with a 16 guage needle to allow for a vacuum within the tubes. The prepared aliquots were frozen at −80° C. prior to lyophilization in a benchtop Labonco lyophilizer overnight. The lyophilized tubes were then stored at −80° C.

All publications and patents mentioned herein are hereby incorporated by reference in their entirety as if each individual publication or patent was specifically and individually indicated to be incorporated by reference.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

1. Banoo, S., D. Bell, P. Bossuyt, A. Herring, D. Mabey, F. Poole, P. G. Smith, N. Sriram, C. Wongsrichanalai, R. Linke, R. O'Brien, M. Perkins, J. Cunningham, P. Matsoso, C. M. Nathanson, P. Olliaro, R. W. Peeling, A. Ramsay and T. D. R. D. E. E. Panel (2006). “Evaluation of diagnostic tests for infectious diseases: general principles.” Nat Rev Microbiol 4 (9 Suppl): S21-31.
2. Basile, A. J., K. Horiuchi, A. J. Panella, J. Laven, O. Kosoy, R. S. Lanciotti, N. Venkateswaran and B. J. Biggerstaff (2013). “Multiplex Microsphere Immunoassays for the Detection of IgM and IgG to Arboviral Diseases.” PLOS ONE 8(9): e75670.
3. Caliendo, A. M., D. N. Gilbert, C. C. Ginocchio, K. E. Hanson, L. May, T. C. Quinn, F. C. Tenover, D. Alland, A. J. Blaschke, R. A. Bonomo, K. C. Carroll, M. J. Ferraro, L. R. Hirschhorn, W. P. Joseph, T. Karchmer, A. T. Maclntyre, L. B. Reller, A. F. Jackson and A. for the Infectious Diseases Society of (2013). “Better Tests, Better Care: Improved Diagnostics for Infectious Diseases.” Clinical Infectious Diseases: An Official Publication of the Infectious Diseases Society of America 57(Suppl 3): S139-S170.
4. Cashman, K. A., K. E. Broderick, E. R. Wilkinson, C. I. Shaia, T. M. Bell, A. C. Shurtleff, K. W. Spik, C. V. Badger, M. C. Guttieri, N. Y. Sardesai and C. S. Schmaljohn (2013). “Enhanced Efficacy of a Codon-Optimized DNA Vaccine Encoding the Glycoprotein Precursor Gene of Lassa Virus in a Guinea Pig Disease Model When Delivered by Dermal Electroporation.” Vaccines {Basel} 1(3): 262-277.
5. Chua, A. C., J. Cunningham, F. Moussy, M. D. Perkins and P. Formenty (2015). “The Case for Improved Diagnostic Tools to Control Ebola Virus Disease in West Africa and How to Get There.” PLoS Neglected Tropical Diseases 9(6): e0003734.
6. Datta, S. A., X. Zuo, P. K. Clark, S. J. Campbell, Y. X. Wang and A. Rein (2011). “Solution properties of murine leukemia virus gag protein: differences from HIV-I gag.” J Virol 85(23): 12733-12741.
7. Dupuy, L. C., M. J. Richards, B. Ellefsen, L. Chau, A. Luxembourg, D. Hannaman, B. D. Livingston and C. S. Schmaljohn (2011). “A DNA vaccine for venezuelan equine encephalitis virus delivered by intramuscular electroporation elicits high levels of neutralizing antibodies in multiple animal models and provides protective immunity to mice and nonhuman primates.” Clin Vaccine Immunol 18(5): 707-716.
8. Engel, N., K. Wachter, M. Pai, J. Gallarda, C. Boehme, I. Celentano and R. Weintraub (2016). “Addressing the challenges of diagnostics demand and supply: insights from an online global health discussion platform.” BMJ Global Health 1(4).
9. Fauci, A. S. (2014). “Ebola-Underscoring the Global Disparities in Health Care Resources.” New England Journal of Medicine 371(12): 1084-1086.
10. Feng, K., E. Divers, Y. Ma and J. Li (2011). “Inactivation of a human norovirus surrogate, human norovirus virus-like particles, and vesicular stomatitis virus by gamma irradiation.” Appl Environ Microbiol 77(10): 3507-3517.
11. Fields, B. N., D. M. Knipe and P. M. Howley (2007). Fields virology. Philadelphia, Wolters Kluwer Health/Lippincott Williams & Wilkins.
12. Fields, B. N., D. M. Knipe, P. M. Howley and D. E. Griffin (2001). Fields virology. Philadelphia, Lippincott Williams & Wilkins.
13. Gaudin, Y., S. Moreira, J. Benejean, D. Blondel, A. Flamand and C. Tuffereau (1999). “Soluble ectodomain of rabies virus glycoprotein expressed in eukaryotic cells folds in a monomeric conformation that is antigenically distinct from the native state of the complete, membrane-anchored glycoprotein.” J Gen Virol 80 (Pt 7): 1647-1656.
14. Heymann, D. L., J. Liu and L. Lillywhite (2016). “Partnerships, Not Parachutists, for Zika Research.” New England Journal of Medicine 374(16): 1504-1505.
15. Jose, J., J. E. Snyder and R. J. Kuhn (2009). “A structural and functional perspective of alphavirus replication and assembly.” Future Microbiol 4(7): 837-856.
15. Kay Richmond, J. and D. J. Baglole (2003). “Lassa fever: epidemiology, clinical features, and social consequences.” BMJ 327(7426): 1271.
16. Nguyen, H. T., N. Madani, H. Ding, E. Elder, A. Princiotto, C. Gu, P. Darby, J. Alin, A. Herschhorn, J. C. Kappes, Y. Mao and J. G. Sodroski (2017). “Evaluation of the contribution of the transmembrane region to the ectodomain conformation of the human immunodeficiency virus (HIV-1) envelope glycoprotein.” Virol J 14(1): 33.
17. O'Heam, A. E., M. A. Voorhees, D. P. Fetterer, N. Wauquier, M. R. Coomber, J. Bangura, J. N. Fair, J.-P. Gonzalez and R. J. Schoepp (2016). “Serosurveillance of viral pathogens circulating in West Africa.” Virology Journal 13(1): 163.
18. Phillpotts, R. J. (2006). “Venezuelan equine encephalitis virus complex-specific monoclonal antibody provides broad protection, in murine models, against airborne challenge with viruses from serogroups I, II and III.” Virus Res 120(1-2): 107-112.
19. Satterly, N. G., M. A. Voorhees, A. D. Ames and R. J. Schoepp (2016). “Comparison of MAGPIX®™ Assays and ELISA for the Detection of Hemorrhagic Fever Viruses.” Journal of Clinical Microbiology.
20. Shoemaker, C. J., et al., “Multiple cationic amphiphiles induce a Niemann-Pick C phenotype and inhibit Ebola virus entry and infection.” PLoS One, 2013. 8(2): p. e56
21. Skwarecki, B. (2014). “Breaking the cold chain: Why ditching refrigerators is a big deal for Africa” http://blogs.plos.org/publichealth/2014/02/20/breaking-cold-chain-ditching-refrigerators-big-deal-africa/.
22. Soares, H. R., R. Castro, H. A. Tomas, A. F. Rodrigues, P. Gomes-Alves, B. Bellier, D. Klatzmann, M. J. Carrondo, P. M. Alves and A. S. Coroadinha (2016). “Tetraspanins displayed in retrovirus-derived virus-like particles and their immunogenicity.” Vaccine 34(13): 1634-1641.
23. Souf, S. (2016). “Recent advances in diagnostic testing for viral infections.” Bioscience Horizons: The International Journal of Student Research 9: hzw010-hzw010.
24. Spackman, E. (2012). “Viral diagnostics: will new technology save the day?” Avian Pathology 41(3): 251-258.
25. Institute of Medicine Forum on Microbial Threats. (2007). “Global Infectious Disease Surveillance and Detection: Assessing the Challenges-Finding Solutions; Detection and Diagnostics.” from https://www.ncbi.nlm.nih.gov/books/NBK52875/.
26. Wang, J., Y. Li and Y. Modis (2014). “Structural models of the membrane anchors of envelope glycoproteins E1 and E2 from pestiviruses.” Virology 454-455: 93-101.
27. Zhang R, Hryc C F, Cong Y, et al. “4.4 Åcryo-EM structure of an enveloped aiphavirus Venezuelan equine encephalitis virus.” The HMO Journal. 2011; 30(18):3854-3863. doi: 10. 1038/emboj.2011.261.

What is claimed is:

1. A thermostable complex, comprising
(a) at least one virus-like particle (VLP) that presents at least one viral glycoprotein antigen on its surface; and
(b) a microsphere or bead that is coupled to the VLP;
wherein the complex is capable of serving as an immunoassay platform for detection of an immune response.

2. The thermostable complex of claim 1, wherein the VLP is a homologous VLP or a heterologous VLP or retroviral core VLP.

3. The thermostable complex of claim 2, wherein the VLP presents a viral glycoprotein antigen from at least one of the following: alphavirus family, arenavirus family, Filovirus family, bunyavirus family, or flavivirus family.

4. The thermostable complex of claim 3, wherein the VLP presents a viral glycoprotein antigen from at least one of the following: Crimean-Congo hemorrhagic fever virus (CCHF), Chikungunya virus (CHIK), Dengue virus (DENV), Eastern equine encephalitis virus (EEEV), Lassa virus (LASV), Marburg virus (MARV), Venezuelan equine encephalitis virus (VEEV), or Western equine encephalitis virus (WEEV).

5. The thermostable complex of claim 2 wherein the retroviral core is a MLV-Gag core.

6. The thermostable complex of claim 1, wherein the microsphere or bead is magnetic.

7. The thermostable complex of claim 6, wherein the magnetic microsphere or magnetic bead is fluorescent.

8. The thermostable complex of claim 7, wherein the microsphere or bead has a characteristic length from about 0.1 μm to about 20 μm.

9. The thermostable VCM complex of claim 8, wherein the length is from about 5 μm to about 6 μm.

10. A method of making a thermostable complex, comprising the following steps:
(a) generating a virus-like particle (VLP) that presents at least one viral glycoprotein antigen on its surface in eukaryotic cell culture via transient expression of DNA constructs encoding structural protein(s) and antigen of interest;
(b) purifying the VLP from culture supernatant and characterizing VLP reactivity against control sera containing antibodies of interest; and
(c) conjugating the purified VLP to a microsphere substrate to generate a thermostable complex.

11. A method for detecting an immune response to at least one antibody in a biological sample from a subject comprising:
(a) providing at least one thermostable VLP-conjugated microsphere (VCM) complex, wherein each thermostable VCM complex comprises: (i) a virus-like particle (VLP) comprising a viral glycoprotein antigen on its surface and (ii) a detectably labeled magnetic microparticle or magnetic bead coupled to the VLP;
(b) contacting the thermostable VCM complex with a biological sample from a subject wherein if present, an antibody from the biological sample binds to the viral glycoprotein antigen presented on the VLP of the VCM complex;
(c) determining an amount of antibodies that bind to viral glycoprotein antigens presented on the VCM complex; and
(d) determining an amount of antibodies that do not bind to viral glycoprotein antigens presented on the VCM complex;
wherein immunodetection of antibodies in the biological sample reflects viral infection in the subject and lack of immunodetection of antibodies in the biological sample reflects no viral infection.

12. The method of claim 11, wherein the viral glycoprotein antigen comprises an antigen from at least one of the following: alphavirus family, arenavirus family, Filovirus family, bunyavirus, or flavivirus.

13. The method of claim 11, wherein the VLP is a homologous VLP or aheterologous VLP or a retroviral core VLP.

14. The method of claim 11, wherein the VLP presents a viral glycoprotein antigen from at least one of the following: Crimean-Congo hemorrhagic fever virus (CCHF), Chikungunya virus (CHIK), Dengue virus (DENV), Eastern equine encephalitis virus (EEEV), Lassa virus (LASV), Marburg virus (MARV), Venezuelan equine encephalitis virus (VEEV), or Western equine encephalitis virus (WEEV).

15. The method of claim 13, wherein the retroviral core is an MLV-Gag core.

16. The method of claim 11, wherein the microparticle or bead is magnetic.

17. The method of claim 16, wherein the magnetic microparticle or magnetic bead is fluorescent.

18. The method of claim 16, wherein the magnetic microparticle or magnetic bead has a characteristic length from about 0.1 μm to about 20 μm.

19. The method of claim 18, wherein the length is from about 5 μm to about 6 μm.

20. A method for detecting the presence of a target antibody in a sample, the method comprising:
(a) contacting the sample with a VLP-conjugated microsphere (VCM) complex, wherein the VCM complex comprises: (i) a virus-like particle (VLP) comprising a viral glycoprotein antigen on its surface and (ii) a detectably labeled microparticle or bead conjugated to the VLP, under conditions such that if the target antibody is present in the sample, it will bind in a detectable fashion to the thermostable VCM; and
(b) detecting whether any target antibody has bound to the VCM.

21. A kit for detecting the presence of a target antibody in a sample, which comprises (a) a virus-like particle (VLP) comprising a viral glycoprotein antigen on its surface and a detectably labeled microparticle or bead conjugated to the VLP; (b) suitable packaging material;
(c) optional control materials; and (d) optional instructions for use of the kit.
